(19) United States
(12) Patent Application Publication
(10) Pub. No.: US 2010/0080840 A1
(43) Pub. Date: Apr. 1, 2010

(54) Hybrid superporous hydrogel scaffold for cornea regeneration

(76) Inventors: Michael Cho, Naperville, IL (US); Arpita Kadakia, Chicago, IL (US); Ali Djalilian, Chicago, IL (US); Richard A. Gemeinhart, Chesterton, IN (US)

Correspondence Address: LICATA & TYRRELL P.C., 66 E. MAIN STREET, MARLTON, NJ, 08053, US

(21) Appl. No.: 12/511,145

(22) Filed: Jul. 29, 2009

Related U.S. Application Data
(60) Provisional application No. 61/085,064, filed on Jul. 31, 2008.

(51) Int. Cl. A61F 2/00; A61K 35/12; C12N 11/02

(52) U.S. Cl. 424/427; 424/93.7; 435/177

(57) ABSTRACT

The present invention features a hybrid superporous hydrogel scaffold for cornea regeneration and a method for producing the same. The hybrid hydrogel is composed of a superporous hydrogen matrix with collagen and cells embedded within the pores of the matrix.

This application claims the benefit of U.S. Provisional Application No. 60/085,064, filed Jul. 31, 2008, which is herein incorporated by reference in its entirety.

### BACKGROUND OF THE INVENTION

The cornea is an avascular and optically transparent tissue that refracts and filters light rays before they enter the eye. A clear cornea is essential for clear vision. The cornea may become opacified following injuries, degenerations, or infections. The Vision Share Consortium estimates that corneal blindness affects more than 10 million patients worldwide (Carlsson, et al. (2003) Curr. Opin. Opthalmol. 14(4):192-7). The gold standard treatment is surgical replacement of the cornea using freshly donated cadaver human corneas. Currently, about 40,000 corneal transplants are performed each year in the United States (Eye Bank Association of America. Statistical report 2000), with a 2-year success rate as high as 90% for uncomplicated first grafts performed in nonvascularized “low-risk” patients (Council on Scientific Affairs (1988) JAMA 259:719; The Collaborative Corneal Transplantation Research Group. (1992)Arch. Opthalmol. 110:1392). However, the success in low-risk corneal transplantation contrasts sharply with the results of corneal grafts placed in so-called “high-risk” patients in which rejection rates can increase up to 50-70%, even with maximal local and systemic immune suppression (Mader & Stulting (1991) Opthalmol. Clin. North Am. 4:411; Foulks & Sanfilippo (1982) Am. J. Opthalmol. 94(5):622-9). Immune-medicated rejection still remains the leading cause of corneal transplant failure (Ing, et al. (1998) Opthalmology 105(10):1855-65). The risk factors for immunologic rejection include previous graft rejection, corneal vascularization, and young age. These “high risk” patients typically undergo repeated surgeries resulting it excessive pain, cost, and use of limited resources. For instance, it is not uncommon for an infant born with corneal disease to undergo 15-20 corneal transplants by the time they reach adulthood with each graft lasting only 3-6 months before being succumbed to rejection. Therefore, there is a distinct need for alternative treatments that circumvent rejection in these high risk patients (Coster & Williams (2003) Eye 17(8):996-1002).

A major advantage of an artificial cornea is the absence of immune-rejection. Two artificial corneas are available for transplantation, the Boston Keratoprosthesis (KPro) and AlphaCor. The Boston KPro pioneered the modern core-and-skirt design in which a biointegrable skirt surrounds an optically clear core (Chirila & Crawford (1996)Gesnerus 53(3-4):236-42). AlphaCor later modified this design by utilizing a soft polymer to avoid complications associated with rigidity of the Boston Kpro. Both devices have high retention rates: AlphaCor reports 92% after 6 months (Hicks, et al. (2006) Cornea 25(9):1034-42) and Boston Kpro indicates 95% after 8.5 months (Zerbe, et al. (2006) Opthalmology 113(10):1779. e1, 1779. e7). However, neither is widely accepted due to a lack of stable host integration which eventually results in melting, extrusion, and rejection (Chirila (2001) Biomaterials 22(24):3311-7). In addition, a lack of epithelialization over the anterior surface renders the eye unprotected and susceptible to infections (Myung, et al. (2007) Biomed. Microdevices 9:911-922). For these reasons, artificial corneas are rarely used in clinical practice and reserved for patients who have failed all standard therapies.

To overcome such limitations, other porous polymers, including polytetrafluroethylene, poly-urethane, poly(2-hydroxyethyl methacrylate) (Carlsson, et al. (2003) supra), and poly(ethylene glycol) (Myung, et al. (2007) supra) have been investigated. While the pores provide a physical pathway for cellular migration from host to implant, they do not provide biological cues for cells to adhere. It is apparent that cells respond differently to extracellular cues presented in a three dimensional (3-D) versus a two dimensional (2-D) context. Cell adhesion is markedly altered in 2-D due to the artificial polarity created by the air-substrate interface. A 3-D extracellular environment is a key component contributing to the success of a tissue engineering scaffold. Despite the evidence encouraging 3-D tissue engineering scaffolds, however, they are largely limited by diffusion capabilities. Therefore a porous system is necessary to facilitate nutrient and waste exchange throughout the construct (Karande, et al. (2004) Ann. Biomed. Eng. 32(12):1728-43; Karageorgiou & Kaplan (2005) Biomaterials 26(27):5474-91). Pores are also advantageous post-implantation where they can serve as conduits for host cell integration. The surrounding tissue, including blood vessels and neurons, can migrate into the scaffold via the interconnected pore network further cementing the construct within the tissue.

Many methods have been employed to engineer 3-D porous scaffolds including salt leaching, freeze drying, and layer by layer lithography using heat, adhesives, light, or molds. While these methods have many advantages, major drawbacks include difficulty in achieving interconnected pores, toxic byproducts, difficulty incorporating cells, or long processing times (Tsang & Bhatia (2004) Adv. Drug Deliv. Rev. 56(11):1635-4).

U.S. Pat. No. 6,960,617 describes the use of hydrogels with improved elasticity and mechanical strength. The hydrogels taught are superporous and are used to form a network of polymer chains. The patent does not teach or suggest combining any other compound with the hydrogel in order to improve the function or biocompatibility of the polymer.

Corneal tissue engineering is challenging because it requires the incorporation of several cell types in distinct layers. The epithelium is the outermost layer of the cornea composed of squamous epithelial cells. The main functions of the epithelium are to block foreign materials from entering the eye, and to absorb oxygen and nutrients for the cornea. Bowman's layer is an acellular sheet of collagen separating the epithelium from the stroma. The stroma, located beneath Bowman's layer, is composed of water, collagen, and keratocytes, and is devoid of blood vessels. Below the stroma lies Descemet's membrane, another acellular layer that separates the stroma from the endothelium. The endothelium is the innermost layer which serves as a pump to regulate the hydration lever of the cornea.

Collagen matrices support cell growth and differentiation (Sun, et al. (2004) Tissue Eng. 10(9-10):1548-57; Yoneno, et al. (2005) J. Biomed. Mater. Res. A 75(3):733-41; Reyes & Garcia (2004) J. Biomed. Mater. Res. A 69(4):591-600). Collagen is a natural component of human extracellular matrix and is the most abundant protein in mammalian tissue. In addition, collagen is non-toxic, biodegradable, and inexpensive. As an extracellular matrix (ECM) protein, collagen provides an array of integrin binding sites for cell adhesion. This allows a two-way stream of communication between the cell and the ECM that mediates many of its mechanical and biological characteristics (Pampaloni, et al. (2007) Nat. Rev. Mol. Cell Biol. 8(10):839-45). Unfortunately, collagen gels created in vitro have long been criticized for their weak mechanical properties. To increase the mechanical stability of collagen, chemical cross-linking or dehydration has been attempted (Drury & Mooney (2003) Biomaterials 24(24):4337-51). However, such methods are often toxic to cells and prevent 3-D encapsulation of cells within the matrix.

Many different types of biosynthetic matrices are described by others. For example, U.S. Patent Application No. 2004/0048796 teaches the use of collagen biofabric for medical and surgical applications. The collagen biofabric is prepared from a placental membrane preferably human, by decellularizing the amniotic membrane. U.S. Patent Application No. 2006/0246113 teaches use of a biosynthetic matrix comprising a hydrogel that is formed by chemically cross-linking a synthetic polymer and a biopolymer. EP 1 741 457 discloses a biosynthetic matrix comprising a hydrogel which is formed by cross-linking a synthetic polymer and a biopolymer as well. However, none of these matrices has been successfully used to produce a corneal implant material with sufficient strength and biocompatibility for use in corneal replacement surgery. There remains a need for scaffold materials that can be used in corneal replacement surgery.

### SUMMARY OF THE INVENTION

The present invention is a hybrid scaffold for cornea regeneration. The hybrid scaffold is composed of a superporous hydrogel matrix with cells and collagen in the pores of said superporous hydrogel matrix. In a preferred embodiment, the cells are fibroblasts. In some embodiments, the hybrid scaffold further includes epithelial cells attached to the surface of said matrix via a layer of collagen. In other embodiments, the hybrid scaffold contains a central core filled with optically clear macromer. A method for producing a hybrid scaffold is also provided.

### BRIEF DESCRIPTION OF THE DRAWINGS

FIG. 1 depicts the production of a hybrid scaffold for cornea regeneration. FIG. 1A shows reswelling of a dehydrated superporous hydrogel (SPH) by immersion into the solution containing collagen and fibroblasts. FIG. 1B shows a rehydrated SPH with cells and collagen entangled within the pores and a central hole introduced into the SPH. FIG. 1C shows nonporous PEGda introduced into the central hole and lining the anterior surface. FIG. 1D depicts the modification of the anterior surface of the SPH with collagen and epithelial cells.

FIG. 2 depicts the in vivo results obtained when the artificial cornea of the present invention is implanted into rats and the corneas are observed after 2 weeks. FIG. 2A shows the results when one rat received the hybrid skirt (SPH and collagen). FIG. 2B shows the results when another rat received an implant of only SPH (not a hybrid). As can be seen clearly when the two figures are compared, the implant with collagen (FIG. 2A) was less noticeable after 2 weeks, indicating that the implant was better integrated with the surrounding tissue as compared to the implant without collagen (FIG. 2B).

### DETAILED DESCRIPTION OF THE INVENTION

A hybrid scaffold composed of collagen intertwined in a polyethylene glycol diacrylate (PEGDA)-based superporous hydrogel (SPH) has now been developed to provide complete 3-D cell adhesion and encourage cell ingrowth, while maintaining the overall mechanical strength of the SPH. The hybrid scaffold of the invention is produced by dehydrating the SPH, then reswelling in a collagen-cell solution to create a hybrid scaffold without covalent bonding or close interactions between the materials. This method was determined to result in better 3-D cell adhesion compared to scaffolds created with intimate entangling of collagen and PEGDA polymer chains. Since cells are embedded entirely within the collagenous portion and not exposed to the walls of the SPH, precise control or uniformity of pore size is unnecessary. Furthermore, since cells can be embedded within collagen prior to uptake and gelation, this method creates a 3-D environment and avoids unnatural cell polarity.

The method of this invention produces a scaffold that has strong bulk properties, yet natural 3-D cell adhesive properties. The natural and synthetic gels of the instant hybrid scaffold are intertwined in a noncovalent, nonadherent fashion. In this regard, the collagen of the hybrid scaffold is not attached to the walls of the superporous hydrogel, thereby allowing the collagen gel to contract. As such, cells embedded within collagen are immersed in 3-D in the collagen gel and are not exposed to the walls of the superporous hydrogel. While collagen increases cell adhesion, retention, and ingrowth, the overall mechanics of the hybrid are not dependent on collagen and greatly resembles the superporous hydrogel. Thus, the hybrid superporous hydrogel provides mechanical stability and interconnected pores while the 3-D collagen matrix provides 3-D adhesive binding sites. While the exemplified hybrid scaffold is composed of collagen and PEGDA, it is contemplated that this versatile method can be adapted to incorporate many different natural and synthetic materials as appropriate to a specific tissue type. Indeed, the instant hybrid scaffold can be used effectively with or without preseeding cells based on the desired application.

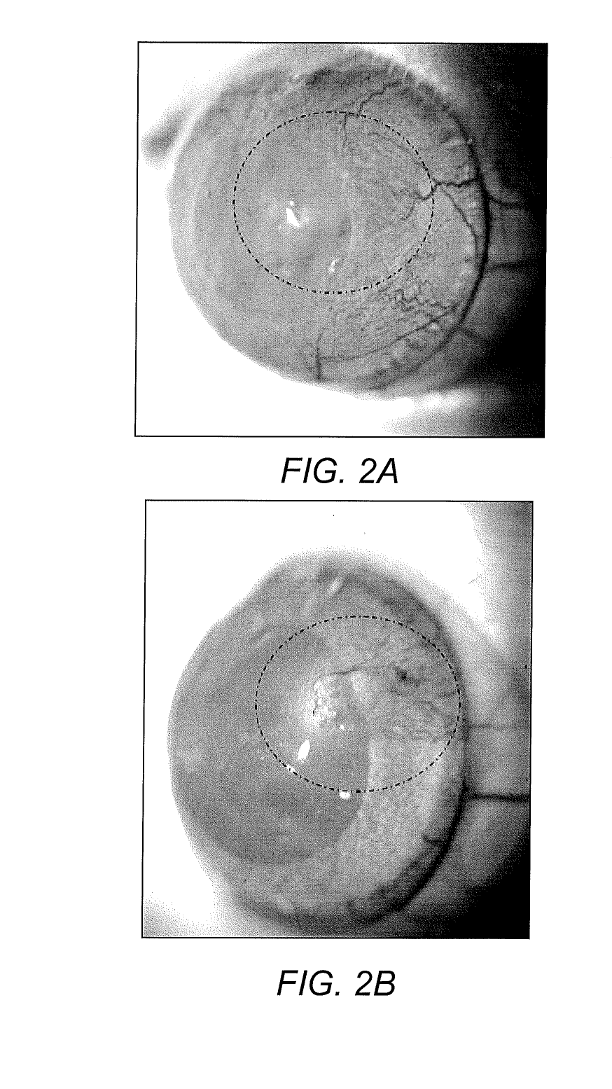

In order to demonstrate that the hybrid scaffolds of the present invention could be successfully used in vivo, two rats were implanted with artificial corneas comprising either the hybrid scaffold (SPH and collagen) or a scaffold of hydrogel without collagen (no hybrid). The novel nature of the artificial corneas produced using the hybrid scaffold of the present invention is related to the hybrid skirt (outer) portion of the implant. Thus, while the central portion of the implant maintains clarity, the peripheral skirt is designed to encourage integration with the tissue of the eye. To test the degree of in vivo integration of this type of corneal implant, a peripheral skirt was implanted into rat corneas and observed after 2 weeks (as shown in FIG. 2). Examination of the photographs in FIG. 2A and FIG. 2B clearly shows that there were differences depending on the nature of the implant. When FIG. 2A is compared with FIG. 2B, it is seen that the implant with a peripheral skirt containing the hybrid material (SPH and collagen) was less noticeable in the eye after 2 weeks. The hybrid skirt implant was well tolerated and biocompatible. Further, the results indicated that the hybrid skirt implant was better integrated with the surrounding eye tissue as compared to the implant without collagen.

Accordingly, the present invention provides hybrid scaffolds composed of collagen and cells incorporated into the pores of a superporous hydrogel and a method for producing the same. A superporous hydrogel matrix, as used herein, refers to a semi-solid three-dimensional structure which is capable of absorbing a substantial amount of water due to the presence of a plurality of inter-connected macropores of average diameter size between about 100 nm and about 300 μm. Superporous hydrogels can be produced as disclosed herein, i.e., a foaming reaction optimized for simultaneous polymerization, or any other suitable method employed in the art. As is appreciated by the skilled artisan, the size of the macropores can be dependent on a number of factors including, e.g., the nature of the solvent or solvents in which the gel is formed and/or the amount of polymerization initiator or catalyst.

“Superporous” is intended to mean that the matrix swells rapidly in solution (<1 minute). In so far as uptake of material is based on capillary action rather than diffusion (Gemeinhart, et al. (2000) J. Biomater. Sci. Polym. Ed. 11(12):1371-80; Gemeinhart, et al. (2001) J. Biomed. Mater. Res. 55(1):54-62), the instant superporous hydrogels can rapidly incorporate a variety of soluble materials, such as cells and proteins, within the pores of the scaffold matrix. Superporous hydrogels are composed of polymers that will swell, without dissolving, when placed in water or other biological fluids. Hydrogels can generally absorb a great deal of fluid and, at equilibrium, typically are composed of 60-90% fluid and only 10-30% polymer. Hydrogels are particularly useful due to the inherent biocompatibility of the cross-linked polymeric network (Hill-West, et al. (1994) Proc. Natl. Acad. Sci. USA 91:5967-5971). Hydrogel biocompatibility can be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). Also, hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36).

Hydrogel matrices of the invention are composed of synthetic hydrophilic polymers which have been synthetically produced and which are hydrophilic, but not necessarily water-soluble. Examples of synthetic hydrophilic polymers which can be used in the practice of the present invention are polyethylene glycol (PEG); polyoxyethylene; polymethylene glycol; polytrimethylene glycols; polyvinylpyrrolidones; poly(acrylic acid); poly(itaconic acid); poly(methacrylic acid); poly(hydroxypropyl acrylamide) (HPMA); poly(peptides) such as polyglutamate, polylysine, polyaspartate, polyserine, polythreonine, polycysteine; and polyoxyethylene-polyoxypropylene block polymers; and copolymers, and derivatives and mixtures thereof. While natural marine biopolymers such as agarose, chitosan, and alginate are also embraced by the present invention, in some embodiments, the hydrophilic matrix is not a naturally occurring polymer such as a protein, starch, cellulose, heparin, or hyaluronic acid.

Although different synthetic hydrophilic polymers and selected biopolymers can be used in connection with forming the hydrophilic matrix of the invention, the polymer must be biocompatible and hydrophilic, but crosslinked physically or chemically to prevent dissolution. Particularly suitable polymers include the various forms of derivatized PEG which are extensively used in the modification of biologically active molecules because they lack toxicity, antigenicity, and immunogenicity; have a wide range of solubilities; are generally non-biodegradable and are easily excreted from most living organisms including humans.

Poly(ethylene glycol) diacrylate (PEGDA) hydrogels have been widely accepted in many biomedical applications (Peppas, et al. (1999) J. Controlled Release 62:81-87). PEGDA hydrogels are hydrophilic, biocompatible, nontoxic, and exhibit variable mesh size depending upon PEG macromer length. As exemplified herein, superporous hydrogels produced from PEGDA are not toxic to cells and can be readily produced using the gas foaming method. Moreover, PEGDA is optically clear rendering it ideal clarity in vivo. Accordingly, particular embodiments of the present invention embrace superporous hydrogels produced with PEGDA.

Superporous hydrogels of the invention can be modified to possess high mechanical strength by incorporating a cross-linked hydrophilic agent such as sodium alginate, pectin, chitosan, or (polyvinyl) alcohol that can crosslink after the matrix is formed (Omidian, et al. (2006) Macromol. Biosci. 6:703-10). Hydrogels can also be made degradable in vivo by incorporating PLA, PLGA or PGA polymers. Moreover, superporous hydrogels can be modified with fibronectin, laminin, vitronectin, or, for example, RGD for surface modification, which can promote cell adhesion and proliferation (Heungsoo Shin (2003) Biomaterials 24:4353-4364; Hwang, et al. (2006) Tissue Eng. 12:2695-706). Indeed, altering molecular weights, block structures, degradable linkages, and cross-linking modes can influence strength, elasticity, and degradation properties of the instant hydrogels (Nguyen & West (2002) Biomaterials 23(22):4307-14; Ifkovits & Burkick (2007) Tissue Eng. 13(10):2369-85).

Superporous hydrogels can also be modified with functional groups for covalently attaching a variety of proteins (e.g., collagen) or compounds such as therapeutic agents. Therapeutic agents which can be linked to the matrix include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent can also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the matrix can be via a protease sensitive linker or other biodegradable linkage.

In addition to functional groups, the polymers of the instant hydrogels can further contain a means for controlled biodegradation to facilitate removal of the matrix polymer from the subject being treated. For example, PEGDA hydrogels can be made to biodegrade at a faster rate by modification (Sawhney, et al. (1994) J. Biomed. Mater. Res. 28:831-838). PEGDA hydrogels can be made biodegradable by incorporating a biodegradable cross linker or by utilizing biodegradable copolymers (Sawhney, et al. (1993) Macromolecules 26:581-587; Park, et al. Biodegradable Hydrogels for Drug Delivery. 1993, Lancaster, Pa.: Technomic Pub. ix, 252; Watanabe, et al. (2002) Biomaterials 23:4041-4048; Yamini, et al. (1997) J. Macromol. Sci. A34:2461-2470). For example, telechelic biodegradable block copolymers, specifically degraded by either plasmin or crude collagenases, have been used in cross-linked hydrogels (West, et al. (1999) Macromolecules, 32:241-244). The extent and rate or degradation is controlled by the specific degradation mechanism used thereby limiting accumulation of the matrix at the site of implantation.

As indicated, hydrogels of the invention can be produced by conventional gas foaming methods, wherein a hydrogel precursor solution is prepared and a foaming agent is added thereto to produce a foam, which gels or polymerizes to form a matrix with a plurality of macropores dispersed therein. A precursor solution is defined as the mixture of components which are combined to produce the superporous hydrogel structure, but lacks a foaming agent which facilitates foam formation and gelling or polymerization of the hydrogel. A precursor solution of the invention can include, but is not limited to, a hydrophilic polymer, an initiator, and a foam stabilizer. Suitable hydrophilic polymers are disclosed herein. Suitable initiators include, e.g., APS/TEMED and a suitable foam stabilizer can be a block copolymer such as PLURONIC F-127. A foaming agent can be a chemical or physical foaming agent. In some embodiments, the foaming agent is sodium bicarbonate. In other embodiments, the foaming agent is a gas, e.g., compressed air or nitrogen. Other foaming agents of use in the gas foaming method are known to those of skill in the art.

In accordance with the method for producing a superporous hydrogel of the invention, the superporous hydrogel matrix is dehydrated. The hydrogel matrix can be dehydrated by any suitable chemical and/or physical means. For example, dehydration can be achieved using a combination of alcohol (e.g., ethanol) and a dehydrator.

To incorporate a molecule of interest (e.g. collagen), with or without cells embedded therein, the hydrogel matrix is reswelled or rehydrated in a solution containing the molecule of interest with or without the cells. Molecules which can be incorporated into the pores of the superporous hydrogel matrix include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents. In certain embodiments, the molecule of interest is collagen. In particular embodiments, the collagen is Type I collagen. Desirably the molecule of interest is in a biologically compatible solution, i.e., a solution which is non-toxic in vivo. Suitable solutions include, but are not are limited to, water, saline, a buffer and the like.

Type I collagen is the most abundant collagen of the human body. It is present in scar tissue, tendons, and the organic part of bone. Type II collagen is a component of articular cartilage and is found in association with Type IX collagen, whereas Type III collagen is the collagen of granulation tissue, and is produced quickly by young fibroblasts before the tougher type I collagen is synthesized. Type XII collagen is found to interact with types I and III collagen. Type IV collagen is part of the basal lamina. Type V and Type VI collagen are components of most interstitial tissue and are associated with type I collagen. Type VII collagen is a component of the epithelia as is Type VIII collagen. Type X collagen is hypertrophic and part of mineralizing cartilage, whereas Type XI collagen is a component of cartilage. Therefore, depending on the site of implantation and the intended therapeutic result, one or more collagens can be incorporated into the pores of the superporous hydrogel matrix. Collagens can be obtained in solution as a pepsin-solubilized collagen dissolved in acid (e.g., Vitrogen; ANGIOTECH® Biomaterials, Palo Alto, Calif.). The collagen can be neutralized (e.g., to pH 7.0 to pH 7.4 with NaOH), and directly incorporated into the superporous hydrogel matrix or combined with a cell of interest and be incorporated into the superporous hydrogel matrix. The collagen can than be solidified via fibrillogenesis (e.g., at 24° C. to 37° C. in the presence or absence of CO2) with cells suspended therein.

When it is desired that cells be incorporated into the instant superporous hydrogels, the cells can be combined with the solution containing the molecule of interest prior or after the solution has be used to reswell or rehydrate the hydrogel. In particular embodiments, the cells are added to the solution prior to reswelling the hydrogel. Types of cells of particular use in this invention include, but are not limited to, stem cells, fibroblasts, epithelial cells, endothelial cells, mesenchymal cells, insulin-producing islet cells, hepatocytes, myocytes, neurons, chondrocytes, skin cells, bone marrow cells, and the like. The cells can be autogenic, allogenic or xenogeneic with respect to the subject receiving the instant hybrid superporous hydrogel. Cells can be isolated from biopsy samples or generated by differentiation and expansion of stems cells using conventional methods. In addition to being incorporated into the pores of the hydrogel matrix, some embodiments embrace encapsulation of cells within the hydrogel itself, e.g., by adding cells to the hydrogel precursor solution prior to polymerization. Cells encapsulated within the hydrogel matrix and hydrogel pores can be the same or different. For example, one could encapsulate stem cells in the hydrogel matrix and encapsulate cells capable of producing growth or differentiation factors in the pores, or vice versa.

In addition to cells incorporated into the pores of the hybrid superporous hydrogel, the present invention further embraces coating one or more surfaces of the hybrid superporous hydrogel matrix with one or more of the cell types disclosed herein. In particular, the present invention embraces attaching epithelial cells to the surface of the hybrid hydrogel matrix via a layer of collagen. In addition, the invention embraces the inclusion of a central core in the hybrid hydrogel which is filled with one or more optically clear macromers. For the purposes of the present invention, a macromer is optically clear when it can transmit light at wavelengths ranging from 200 nm to 1000 nm and has a reflective index of more than 1 or more desirably more than 1.3. Suitable macromers include, e.g., the hydrophilic polymers disclosed herein. In particular embodiments, the optically clear macromer is PEGDA.

As demonstrated herein, the components and fabrication method of the invention are not toxic to cells and enables the incorporation of cells within the pores of the superporous hydrogel. Such hydrogels find application as biological scaffolds for maintaining and growing cells and in the functional replacement of injured or damaged organs of the body. In certain embodiments, the instant hybrid superporous hydrogel is used in the preparation of a variety of formed implants for use in medical applications. Advantageously, the superporous hydrogel is designed to provide cells to a damaged or injured site to facilitate regeneration. Accordingly, the instant composition is useful for providing localized delivery of cells to a subject. Such delivery can be used to, e.g., promote wound healing and in tissue regeneration or replacement. In particular embodiments, the hydrogels of the present invention are used in tissue engineering or regenerative medicine, as a model organ system for drug testing, or for use in cell expansion.

In particular embodiments of this invention, the instant hybrid superporous hydrogel is used in the preparation of an artificial cornea. In this regard, specific embodiments embrace the incorporation of collagen and corneal fibroblasts into the pores of the superporous hydrogel.

Depending on the application, the superporous hydrogel of the invention can be used alone or in admixture of a pharmaceutically acceptable carrier in a pharmaceutical composition. Suitable formulations for use in the present invention are found in Remington: The Science and Practice of Pharmacy, Alfonso R. Gennaro, editor, 20th ed. Lippincott Williams & Wilkins: Philadelphia, Pa., 2000. Exemplary carriers include, e.g., water, saline, a buffer and the like. The compositions can also contain pharmaceutically acceptable auxiliary substances as required to approximate physiological conditions, such as pH adjusting and buffering agents, tonicity adjusting agents, wetting agents, detergents and the like.

The compositions of the invention can be formulated for any appropriate manner of administration, including for example, topical, subcutaneous implantation or intramuscular implantation depending on the site at which cells are to be delivered and the disease or condition be treated.

The present method of producing a hybrid superporous scaffold is simple, inexpensive, and versatile. Therefore, it can be applied to many tissue engineering applications including skeletal and soft tissue applications. For example, in addition to corneal regeneration, the hybrid superporous hydrogen can be used in bone tissue engineering. Indeed, many modifications can be made to the disclosed hydrogel to tailor it for a particular tissue. Hydrolytic linkages can be incorporated within the SPH to create degradable and non toxic by products over time. Drugs or molecules could be loaded within the SPH for controlled release situations. Both natural and synthetic materials can be altered to produce specifically desired properties. For example, the current PEGDA-based SPH cannot withstand tensile forces including suturing. Therefore another elastic material could be used in place of PEGDA if suturing is necessary.

The invention is described in greater detail by the following non-limiting examples.

### Example 1

### Materials & Methods

Cell Culture. Two cell types, stem cells and committed cells, were analyzed. Human mesenchymal stem cells (MSCs) were maintained in Gibco's α-Minimal Essential Medium (with L-glutamine, without ribonucleosides, without deoxyribonucleosides) containing 15% fetal bovine serum (FBS), 1% L-glutamine, and 1% antibiotics. The HT-1080 human fibrosarcoma cell line was purchased from ATCC (Manassas, Va.). Fibroblasts were bathed in Dulbecco's Modified Eagle's Medium (DMEM) supplemented with 10% fetal bovine serum (FBS) and 1% antibiotics/antimycotics. Media was changed every two to three days to remove wastes and provide fresh nutrition. Cells were maintained at 37° C. in the presence of 5% CO2 and 95% air. Cells were plated at a density of 3×103 cells/cm2 in tissue culture flasks until a 75-80% confluent monolayer was formed. Cells were passaged by incubating for 5 minutes with 0.25 mg/mL trypsin and replating at the above density. All cells used in the experiments herein were between passages 3 and 6. The method presented herein can be extended to other cell types. Cells can be either loaded into the scaffold prior to implantation or cells can be encouraged to migrate into the scaffold post-implantation.

Collagen Gel Preparation. Rat tail collagen type I (BD Biosciences, San Diego, Calif.) was mixed with 0.1 N NaOH, 10× Hank's Balanced Salt Solution (HESS), and 0.1 N acetic acid at a volume ratio of 3:2:1:1. This created a neutral pH collagen solution at a concentration of 1 mg/mL. If cell seeding was desired, cells were suspended in the collagen solution at a concentration of 1 millions cells/mL to encapsulate them in a 3-D network. Soaking a dehydrated SPH in this solution allowed uptake of cells and collagen within the pores. Collagen gelation was initiated by warming to 37° C. for 30 minutes. If pre-seeding with cells was not desired, the SPH was soaked in the collagen solution without cells. Again gelation occurred by warming to 37° C. for 30 minutes.

Superporous Hydrogel Fabrication. A 20% (w/v) PEGDA solution (500 μL) was combined with the following reagents: 60 μL of 10% pluronic PF-127, 30 μL of 20% Temed, 20 μL of acrylic acid, and 23 μL of APS. The final volume was adjusted to 1 mL via addition of deionized water. The solution was heated for two minutes at 37° C. Subsequently, 200 mg of sodium bicarbonate was mixed in the solution, which created a foaming reaction resulting in a porous structure. The amount of sodium bicarbonate was varied from 100 to 300 mg to create differences in pore architecture. SPHs were rinsed in water to remove unreacted sodium bicarbonate crystals. To prevent pore collapse, the scaffolds were dehydrated in ethanol overnight. Scaffolds were then further dehydrated in a food dehydrator for 45 minutes. Cut sections were placed under UV light for 20 minutes to sterilize.

Pore Architecture and Swelling Measurements. A scanning electron microscope was used to picture the pore architecture of the SPH. SPHs made with varying amounts of sodium bicarbonate were imaged. Rapid swelling to large volumes is an important feature for this application. A swelling ratio, Q, was determined by comparing the mass of the swollen SPH to the mass of the dehydrated SPH. Dehydrated structures of varying pore sizes were soaked in water for at least 20 minutes. All SPHs were centrifuged at 1000 rpm for 3 minutes to remove air bubbles. SPHs were strained with a sieve, to remove excess water, and weighed. This mass represents the water accumulated in the pores as well as in the hydrogel structure itself. Subsequently, the SPHs were gently squeezed and blotted to remove water in the pores but maintain water in the hydrogel structure. By dividing the swollen weight by the initial weight, two swelling ratios, Q_{Total Water} & Q_{Hydrogel Water}, were obtained.

Q_{Total Water}=Weight_{Total Water}/Weight_{Dehydrated}

Q_{Hydrogel Water}=Weight_{Hydrogel Water}/Weight_{Dehydrated}

Cell Staining. SPH constructs with and without collagen were employed as 3-D fibroblast scaffolds. Cells were loaded as described above and incubated for 24 and 48 hours. A focal adhesion kit (Chemicon, Temecula, Calif.) was used to visualize cell adhesion and retention. Rhodamine phalloidin stained microfilaments red and DAPI stained nuclei blue. A live/dead viability kit (Molecular Probes, Eugene, Oreg.) was used to show cells that were alive (green) versus dead (red). A Bio-Rad confocal microscope was used to image each of these structures.

Cell Migration. In cases where pre-seeding with cells is not desirable, it was determined whether the hybrid scaffold was preferred for cell migration into the scaffold. Acellular SPH scaffolds with and without collagen were placed atop a monolayer of cells. Cell migration into the scaffold was monitored over 3 weeks. Cells were stained with live/dead viability and visualized with a confocal microscope.

Compressive Measurements. Compressive modulus of the SPH scaffolds was determined by compressive testing. Water swollen SPHs were sandwiched between two pieces of glass lined with Velcro (to prevent slippage) and compared to collagen swollen SPHs. Incremental weights were placed atop and the amount of strain that each SPH withstood was recorded. A stress versus strain curve was plotted to determine an estimate of compressive modulus.

Central Optic Hydrogel Synthesis. A hydrogel solution was prepared by mixing 5% (w/v) of PEGda in sterile PBS. A photoinitiator, Igracure 2959 (Ciba, Tarrytown, N.Y.) was added to the PEGDA solution for a final concentration of 0.025% w/v. Cell viability was assessed in response to photoinitiator, UV light exposure, and PEGda concentration. Igracure 2959 was the least toxic photoinitiator to mammalian cells. A concentration of 0.03% (w/v) or less is considered optimal (Williams, et al. (2005) Biomaterials 26(11):1211-8). Placing this solution under UV light (365 nm, 4 mW) for 10 minutes created a clear polymerized gel. The gel was soaked in fresh PBS to remove unreacted monomers and initiator.

### Example 2

### Swelling Ratio

Since collagen begins to gel quickly after pH neutralization, immediate upload into the SPH was necessary to facilitate uniform distribution throughout the SPH. Since the SPH fabrication method created interconnected macrosized pores, swelling occurred in less than 1 minute. Soaking the SPH in a collagen solution allowed natural materials to enter the pores easily and rapidly via capillary action. Thus, wherein preceeding with cells is desired, cells can be suspended in the collagen solution just prior to uptake. Swelling was determined by the degree and size of interconnected pores. SEM analysis of pore structure in three SPHs created with 100, 200, and 300 mg of sodium bicarbonate revealed two types of pores: larger pores which appeared similar in size and shape in each of the SPHs and smaller pores, which formed the interconnection pathways. It was apparent that increasing the amount of sodium bicarbonate resulted in an increased number of interconnection pores. This indicated that the differences in pore architecture caused differences in swelling ratios. Swelling ratios, Q, were determined for SPHs of varying pore sizes (i.e., using 100, 200 or 300 mg sodium bicarbonate). This analysis indicated that QTotal Water increased as more sodium bicarbonate was used. However, QHydrogel Water had no appreciable difference with different pore sizes. This indicated that differing amounts of sodium bicarbonate altered pore structure, but the amount of hydrogel in each SPH remained the same. This phenomenon is important to note for applications in which it may be desirable to load molecules within the hydrogel structure itself. In addition, QTotal Water hovered around 100, indicating that the SPH was capable of incorporating about 100 times its dried weight. Therefore, any long term increases in weight due to cell proliferation or ECM production should not be barriers to long term stability.

Rapid uptake into the SPH did not incur cell injury. Indeed, a live/dead viability stain performed one day after cell loading showed that MSC cells were alive and spreading. Calcein AM crossed the cell membrane of live cells and fluoresced green while ethidium homodimer only entered dead cells and fluoresced red. Minimal dead cells were detected. This promised to be an effective and efficient method of cell loading that did not require the use of external forces or compromise cell viability.

While it has been suggested that a small pore size within a narrow range is essential to 3-D cell behavior within scaffolds, the results herein indicate SPH hybrid technology eliminates the need for precise control of pore size or shape with respect to 3-D cell adhesion. The instant method is unique in that noncovalent binding and a lack of intimate contact between scaffold materials separates the cellular microenvironment from the supporting SPH. When cells are in contact with PEGDA, despite the presence of collagen, cells are not able to spread out. Therefore spatial and temporal separation of the two materials is necessary for optimal cell behavior.

As evident in SEM images of the pore structure, shape and size are non uniform. However, observation of cell morphology and adhesion in the SPH-collagen gel showed similarity to purely 3-D collagen images. For that reason SPH pore structure is not a factor in cell behavior because cells do not contact the SPH. Cells in the hybrid matrix appear to be only embedded in the collagenous portion. So long as the pores are interconnected to assure uniform distribution and effective nutrient and waste diffusion, the instant hybrid superporous hydrogel does not require the stringent requirements of other systems to create a natural 3-D cell microenvironment. Thus, the hybrid hydrogel of this invention is more convenient and better mimics natural living systems that generally lack the uniformity imposed by engineered constructs.

### Example 3

### Adhesion Staining

In preseeded scaffolds, it was observed that collagen encouraged fibroblast spreading in 3-D and formation of stress fibers. Scaffolds without collagen housed clumped, round cells that were incapable of attaching to the scaffold. PEGDA is intrinsically resistant to adhesion. Thus, a lack of ECM cell binding sites in non-collagenous scaffolds was presumed to be responsible for the round morphology. After 48 hours, scaffolds without collagen were completely acellular. Having nothing to attach to, cells tended to migrate out of the scaffold and attach to the tissue culture plate below.

In contrast, collagen loaded scaffolds showed cell retention within the scaffold and few if any cells attached to the plate below. Collagen within the hydrogel pores greatly enhanced cell spreading and retention in a 3-D manner. The microfilament stress fibers were clearly observed, indicating that cell adhesion was mediated by integrin binding sites available in collagen, leading to formation of focal adhesion. Corneal fibroblasts have been shown to express a variety of β1 family integrins, which can bind to collagen. In scaffolds without collagen, β1-integrin was distributed uniformly around the periphery of the cell. However, in collagen-filled scaffolds, the β1-integrins were punctuated and clustered at sites of focal adhesions. Addition of antibodies against β1-integrins prevented cells from attaching and spreading on the collagens.

In addition to enhancing cell spreading and retention when embedded in the collagen of the pores, it was demonstrated that this hybrid scaffold also enhances surrounding cell migration into the scaffold by virtue of an open pore structure and collagen binding. Acellular SPH scaffolds with and without collagen were placed on top of a monolayer of fibroblasts. Over 3 weeks, tremendous cellular ingrowth was observed into scaffold with collagen. The scaffold without collagen remained acellular. This demonstrated that pores alone are not sufficient for cellular ingrowth, and the incorporation of collagen greatly enhances this scaffold as an ideal tissue engineering scaffold. Good cell ingrowth is necessary for in vivo implantation so that host cells can migrate into the scaffold and form a strong integration with the surrounding tissue. This is also a conduit for nerve and blood vessel ingrowth which may be necessary for long term survival of the implant.

### Example 4

### Mechanical Measurements

Compressive tests to determine compressive modulus indicated that the SPH was significantly more compressive than nonporous PEGDA alone. The average compressive modulus for the SPH without collagen was 1 kPa. When compared to nonporous PEGDA, it was observed that the SPH was able to withstand much higher compressive forces. This may be of importance in situations where the implant is subjected to high compressive forces. The addition of collagen in the SPH did not have a significant impact on the bulk modulus. Therefore, the instant hybrid hydrogel can maintain a high compressive modulus overall, without subjecting embedded cells to these conditions, since they are only exposed to the much softer collagen microenvironment.

### Example 5

### Optical Properties

The central optic of an artificial cornea should be clear and have an appropriate refractive index. To demonstrate the use of PEGDA in the central core of an artificial cornea, qualitative and quantitative analyses of a 5% PEGda was carried out. For qualitative analysis, written text was viewed with and without an overlying hydrogel. Optical properties such as light transmission and refractive index of the central core were determined using a UV-Vis spectrophotometer and refractometer, respectively. The percent of light transmittance was measured in reference to PBS at wavelengths ranging from 200 nm to 1000 nm. All measurements were made in triplicate. The refractive index of the central optic is measured using a refractometer.

The results of these analyses indicated that a 5% hydrogel yielded excellent optical properties for use as a central optic. For example, clarity of written text viewed with or without an overlying hydrogel was observed to be similar. A quantitative study using UV-Vis spectrophotometry revealed high light transmittance over a broad range of wavelengths. For example, the average transparency at 550 nm was 90%. In addition, the refractive index was approximately 1.34 (˜5 brix) which is only slightly less than the natural cornea, 1.37.

### Example 6

### Artificial Cornea Fabrication

Based on the natural corneal architecture, the anterior surface of a hybrid hydrogel matrix is coated with epithelial cells to encourage host epithelialization to regenerate the protective and nutrient absorbing qualities of the epithelium. Below the epithelium, similar to Bowman's layer, a thin layer of nonporous PEGda is used to separate the epithelium from the underlying stroma. PEGda discourages cell binding and keeps cell types localized. Within the stromal skirt, collagen and cells are surrounded by PEGda, a hydrogel that is capable of retaining large amounts of water to maintain an appropriate shape and hydration level. The hybrid superporous skirt is designed to allow maximal host cell integration through the pores and attachment to cell adhesion sites. The central core is kept free of collagen to maintain optical transparency.

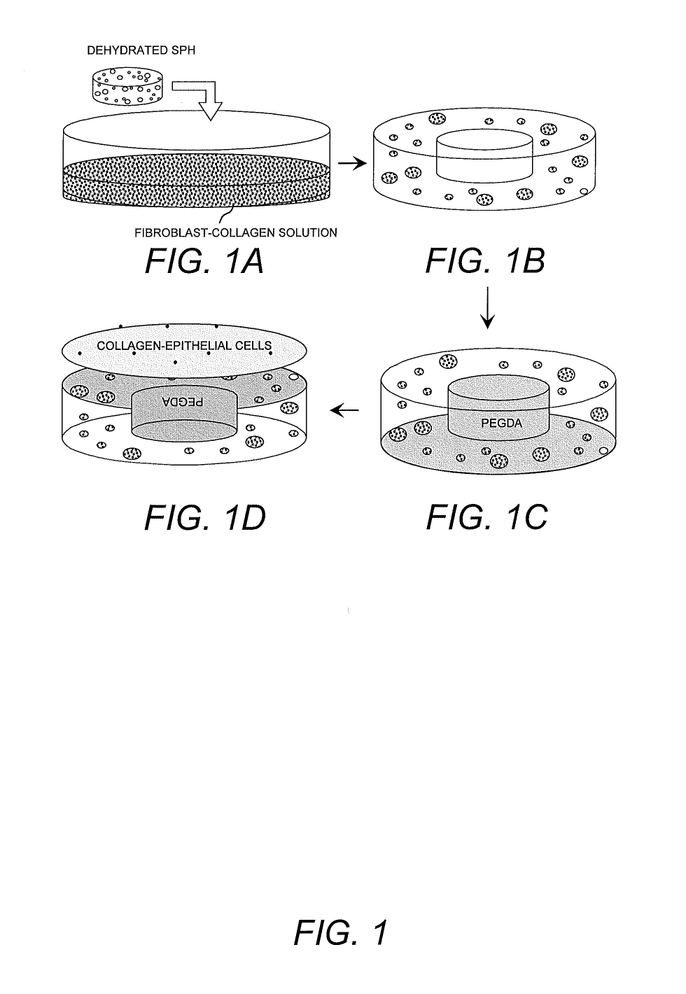

The production of a biodegradable, porous, cell-based, tissue-engineered cornea is depicted in FIG. 1. As a first step in producing such an artificial cornea, an SPH disc was soaked in a fibroblast-collagen solution (FIG. 1A). As a control, SPHs were also soaked in cell solutions without collagen. Submersion in liquid caused rapid swelling of the SPH and uptake of collagen and cells within the pore network (FIG. 1B). Collagen fibers were then thermogelled. The result was a collagenous microenvironment dispersed throughout a mechanically stable hydrogel. A central hole was carved out with a 5 mm trephine (FIG. 1B). The central hole was filled with a nonporous, optically clear PEGda macromer solution (FIG. 1C). The nonporous PEGda solution diffused into the immediate periphery and spread along the bottom surface of the SPH and deposited a thin layer of nonporous PEGda on the anterior surface. The nonporous PEGda was photopolymerized into this irregular shape. The anterior surface could then be modified with collagen so that epithelial cells could be attached for proliferation on the top surface (FIG. 1D).

To epithelialize the surface of the hybrid hydrogel, a water soluble heterobifunctional cross-linker such as sulfo-SANPAH (PierceNet) can be used to attach collagen type 1 to the surface of PEGda. The N-hydroxysuccinimide group attaches to collagen proteins while the phenyl azide group photoreactively inserts into PEGda. The presence of covalently bound collagen can be imaged with second harmonic generation using a multiphoton microscope. Compared to the stroma, the collagen on the surface will be a thin layer ( 1/10 of the concentration). This thin layer of collagen is expected to support the growth of corneal epithelial cells as determined by, e.g., intermediate filament keratin-3 and keratin-12 expression. It is contemplated that this layer can be optimized for sufficient cell attachment while maintaining clarity. Clarity of the collagen layer can be assessed via UV-Vis spectrophotometry.

Corneal epithelial cultures can obtained from rabbit limbal tissue of approximately 3×2 mm at the time of surgery. The tissue is treated with dispase (10 mg/ml) at 4° C. overnight to disrupt the basement membrane. The epithelial sheets are peeled off and digested in 0.25% trypsin-EDTA at 37° C. for 5-10 minutes. Cells are washed and resuspended in keratinocyte serum-free medium (KSFM, Invitrogen), and plated on collagen-coated tissue culture plates. When cells reach 80% confluency, epithelial cells are trypsinized and plated on the hybrid scaffold.

Five major forces have been shown to act on a contact lens (i.e., atmospheric pressure, hydrostatic pressure of the postlens tear film, surface tension of the prelens tear film, lens weight and lid force (Leonardi, et al. (2004) Invest. Opthalmol. Vis. Sci. 45(9):3113-7). Thus, it can be assumed that an artificial cornea will be exposed to similar forces post-implantation. Based on this rationale, assessment of the mechanical properties of the superporous hybrid scaffold can be determined. For example, the Young's modulus can be measured using an atomic force microscope (AFM) and the Hertz model.

The Young's modulus is calculated using Hertz' model:

$${F}_{sphere} = \frac{4}{3} \cdot \frac{E}{( 1 - {v}^{2} )} \cdot {δ}^{3 / 2} \cdot \sqrt{R} = k \cdot d$$

where F is indenting force; R is attached bead radius; δ is indentation, assuming that δ<<R; E is Young's modulus, ν is Poisson ratio (0.5 for incompressible sample); k is cantilever's spring constant, and d is cantilever's deflection. This model assumes a homogeneous, isotropic, semi-infinite elastic material. Also the surface should be flat, a conical or spherical tip should be used, and the indenter material should be much stiffer than the sample.

The Young's modulus of a human donor cornea was reported as 1.3 MPa (Wollensak, et al. (2003) J. Cataract. Refract. Surg. 29(9):1780-5). The Young's modulus of the individual components of the artificial cornea were measured and found to be 2 MPa for a 5% PEGda gel and ˜1 kPa for a 1 mg/mL collagen gel. However, the contribution of collagens to the overall mechanical properties is expected to be minimal.

In addition, the shear modulus of a hybrid scaffold can be determined using elastography. Elastography is a magnetic resonance-based technique that measures mechanical properties by propagating an electromagnetic wave through the material (Zerbe, et al. (2006) supra). The system is ideal for mechanical measurements of soft tissues and complements the AFM measurements. This non-destructive 3D imaging technique can also measure the diffusion of water, which is a direct indication of the tissue structure and viability. When cells swell or cell membranes rupture, for example, water diffusion is more rapid due to fewer physical barriers.

### Example 7

### Biocompatibility of an Artificial Cornea in a Rabbit Model

In vivo biocompatibility in a rabbit model is used to evaluate the degree of host-prosthesis integration, epithelialization, stability, and clarity of the artificial cornea. It is expected that pre-seeding the porous skirt with collagen and fibroblasts will significantly enhance the rate and degree of integration and lead to long-term stability of the implant.

The first step prior to implanting the artificial cornea is to obtain autologous corneal fibroblasts from the rabbit. This is done through a small corneal biopsy from the peripheral cornea. The corneal biopsy tissue is then digested in 1 mg/ml of collagenase overnight at 37° C. The cells are washed and then plated in DMEM supplemented with 10% FCS (Sigma-Aldrich, St. Louis, Mo.), 4 mM L-glutamine and 1% antibiotic solution (Invitrogen-Gibco). After a period of 10-14 days in culture, the fibroblasts are trypsinized and dispersed in collagen solution and incorporated into the superporous skirt of the artificial corneas.

The artificial corneas either seeded with fibroblasts or un-seeded (control) are surgically implanted into the rabbit cornea in a two stage procedure. In the first stage the artificial cornea is implanted as a partial thickness replacement keeping the anterior cornea of the rabbit as a protective flap. Specifically, using a Moria microkeratome (designed to make LASIK flaps) a 130 μm flap of approximately 10 mm diameter is created. This in effect slices the cornea horizontally. The posterior aspect of the cornea which is now exposed is trephinated using an 8 mm trephine and the corneal implant composed of a 4 mm core and 2 mm skirt is sutured in place using interrupted 10-0 nylon sutures. The control rabbits receive the same implants but without any cells imbedded in the skirt. The anterior flap is placed back on top of the implant and sutured to the peripheral cornea using interrupted dissolvable 10-0 vicryl sutures.

In stage two, the portion of the anterior flap which covers the clear zone of the implant is removed and the artificial cornea functions as a full-thickness replacement. The rationale for this staged procedure is to maintain the integrity of the cornea while allowing time for integration to take place. Specifically, animals are returned to the operating room 2 months after the initial implantation. The rabbits are placed under general anesthesia and the central 4 mm of the anterior flap covering the clear zone of the implant is trephinated and removed.

Follow-ups will be performed daily on each rabbit for the first week after surgery and then 2-3 times a week for evidence of complications such as melting, aqueous leakage, extrusion, infection, retroprosthetic membrane formation, retinal detachment, or proliferative vitreoretinopathy. Examinations will include slit lamp biomicroscopy to ensure that corneas are optically clear, and sodium fluorescein staining to assess integrity and barrier function. Intraocular pressure measurements are also taken to determine if the implants are interfering with aqueous humor flow. Indirect opthalmoscopy is used to examine the posterior segment. All animals are followed for 6 weeks to determine the short term outcome and then up to 6 month for long-term studies.

The bio-integration and bio-compatibility of the artificial corneas will be evaluated histologically at 1 week, 2 weeks, 6 weeks, 3 months, and 6 months. Three pairs of rabbits (one experimental and one control) will be used for histopathology at each time point. The eyes will be subjected to routine histology and immuno-staining to evaluate the degree of epithelialization over the core, fibroblast ingrowth into the skirts, and capsule formation around the device. Immunostaining against smooth muscle actin will be used to identify fibroblasts in the superporous skirt. The number and extent of fibroblast penetration into the implant will be graded by masked observers using serial sections starting from the periphery towards the center. The expression of collagen type I, β1-integrin and focal adhesion complexes will likewise be evaluated. The results will be compared between the two groups.

The integration of the artificial corneas will be mechanically tested according to convention methods (Lee, et al. (2000) Arch. Opthalmol. 118(12):1673-8). These measurements will be performed on intact eyes that are enucleated after euthanasia. The intraocular pressure will be progressively increased inside and the pressure at which the host-prosthesis begins to leak will be recorded. These measurements will be done three pairs of eyes (one control, one experimental) for each time point starting at 6 weeks, then 3 months, and 6 months.

As with any prosthetic device, there is a possibility of non-integration with secondary tissue necrosis or extrusion. It is contemplated that constructs pre-seeded with cells will significantly enhance integration. Alternative strategies to promote integration include embedding the skirt with sustained release growth factors such as TGF-beta to promote fibrovascular ingrowth. Another potential problem is membrane formation around the device especially behind the core (Hicks & Hamilton (2005) Cornea 24(6):692-8). In clinical settings, these membranes can typically be removed by YAG laser, however additional strategies for surface modification may also be used to inhibit membrane formation.

What is claimed is:

1. A hybrid scaffold for cornea regeneration comprising a superporous hydrogel matrix with cells and collagen in the pores of said superporous hydrogel matrix.

2. The hybrid scaffold of claim 1, wherein said cells are fibroblasts.

3. The hybrid scaffold of claim 1, further comprising epithelial cells attached to the surface of said matrix via a layer of collagen.

4. The hybrid scaffold of claim 1, further comprising a central core filled with optically clear macromer.

5. A method for producing a hybrid scaffold comprising dehydrating a superporous hydrogel matrix and reswelling the superporous hydrogel in a collagen-cell solution so that the collagen and cells are embedded into the pores of the superporous hydrogel matrix thereby producing a hybrid scaffold.
